(19) United States
(12) Patent Application Publication
(10) Pub. No.: US 2012/0052558 A1
(43) Pub. Date: Mar. 1, 2012

(54) Flow Controlled Microfluidic Devices

(75) Inventors: Mikhail Sergeevich Chivilikhin, St. Petersburg (RU); Lev Lvovitch Kuandykov, St. Petersburg (RU)

(73) Assignee: CORNING INCORPORATED, Corning, NY (US)

(21) Appl. No.: 13/318,496

(22) PCT Filed: May 27, 2010
(86) PCT No.: PCT/US2010/036333

§ 371 Date: Nov. 2, 2011

(30) Foreign Application Priority Data
May 29, 2009 (RU) 2009120627

(51) Int. Cl. C12M 1/00; B01J 19/00

(52) U.S. Cl. 435/283.1; 422/129; 422/240

(57) ABSTRACT

A microfluidic device (10) comprises at least one reactant passage (60) defined within a layer (50) of the microfluidic device (10) and comprising one or more chambers (70, 75) disposed along a central axis (110). Each chamber (100) is divided at a flow-splitting region (150) into two subpassages (140, 145) that diverge from the central axis (110) and then converge together at a flow-joining region (160). The flow-splitting region (150), the flow-joining region (160) or both may comprise at least one flow-directing cape (180, 185) comprising a terminus (190, 195) positioned along the central axis (110). In some embodiments, each subpassage (140) may comprise at least one bend (170). In other embodiments, each subpassage (310) may comprise at least two spaced bends (330, 335).

### BACKGROUND

The present disclosure is generally directed to microfluidic devices and, more specifically, to microfluidic devices having certain passages therein.

Microfluidic devices, which may be referred to as microstructured reactors, microchannel reactors, microcircuit reactors, or microreactors, are devices in which a fluid can be confined and subjected to processing. In some applications, the processing may involve the analysis of chemical reactions. In other applications, the processing may involve chemical, physical, and/or biological processes executed as part of a manufacturing or production process. In any of these applications, one or more working fluids confined in the microfluidic device may exchange heat with one or more associated heat exchange fluids. In any case, the characteristic smallest dimensions of the confined spaces for the working fluids are generally on the order of 0.1 mm to 5 mm, desirably 0.5 mm to 2 mm.

Microchannels are the most typical form of such confinement, and the microfluidic device may operate as a continuous-flow reactor. The internal dimensions of the microchannels provide considerable improvement in mass and heat transfer rates. Microreactors that employ microchannels offer many advantages over conventional-scale reactors, including vast improvements in energy efficiency, reaction speed, reaction yield, safety, reliability, scalability, etc. The microchannels may be arranged, for example, within a layer that is a part of a stacked structure such as the structure shown in FIG. 1. In FIG. 1, a stacked microfluidic device 10 may comprise a layer 50, in which reactant passages comprising microchannels may be positioned.

According to one embodiment of the present disclosure, a microfluidic device 10 is provided. The microfluidic device 10 may comprise at least one reactant passage 60 defined within a layer 50 of the microfluidic device 10. Each reactant passage 60 may comprise at least one chamber 70, 75 disposed along a central axis 110. Each chamber 100 may comprise a chamber inlet 120 disposed along the central axis 110, a chamber outlet 130 disposed along the central axis 110, and two subpassages 140, 145 disposed between the chamber inlet 120 and the chamber outlet 130. Each subpassage 140, 145 may define a path that diverges from the central axis 110 and then converges toward the central axis 110. Each chamber 100 may comprise further a flow-splitting region 150 disposed between the two subpassages 140, 145 and the chamber inlet 120, such that the flow-splitting region 150 divides the chamber inlet 120 into the two subpassages 140, 145. Furthermore, a flow-joining region 160 may be disposed between the two subpassages 140, 145 and the chamber outlet 130, such that the flow-joining region 160 merges the two subpassages 140, 145. The flow-splitting region 150 may comprise at least one flow-directing cape 180 disposed opposite the chamber inlet 120 and comprising a terminus 190 positioned along the central axis 110. The flow-joining region 160 may comprise at least one flow-directing cape 185 disposed opposite the chamber outlet 130 and comprising a terminus 195 positioned along the central axis 110. It is contemplated that one or both of the flow-splitting 150 or flow-joining 160 regions may include a flow-directing cape as described below.

In further embodiments, the terminus 515, 525, 535, 545, 555, 565 of each flow-directing cape 510, 520, 530, 540, 550, 560 may be curved, straight, stepped, or any combination of these.

In still further embodiments, each subpassage 140 of each chamber 100 may comprise at least one bend 170. Each bend 170 may define a shape configured to change the direction of fluid flow within the subpassage 140 by at least 90°.

In still further embodiments, each subpassage 310 of each chamber 300 may comprise at least two bends 330, 335. The subpassage 310 may comprise a straight region 315 disposed between any two bends 330, 335. The straight regions 315, 325 of the two subpassages 310, 320 may comprise a substantially equal width.

These and additional features by the embodiments of the present disclosure will be more fully understood in view of the following detailed description, in conjunction with the drawings.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 is a schematic perspective view showing a general layered structure of a microfluidic device according to embodiments of the present disclosure;

FIG. 2 is a cross-sectional plan view of vertical wall structures defining a reactant passage according to embodiments of the present disclosure;

FIG. 3A is a plan view of a chamber within a reactant passage of a layer of a microfluidic device according to embodiments of the present disclosure;

FIG. 3B is an inset view of a flow-splitting region of the chamber depicted in FIG. 3A according to embodiments of the present disclosure;

FIG. 3C is an inset view of a flow-joining region of the chamber depicted in FIG. 3A according to embodiments of the present disclosure;

FIG. 4 is a schematic perspective view of a single reactant passage of a layer of a microfluidic device, the passage comprising multiple successive chambers of the type shown in FIG. 3A, according to embodiments of the present disclosure;

FIG. 5A is a plan view of a chamber within a reactant passage of a layer of a microfluidic device according to embodiments of the present disclosure;

FIG. 5B is a schematic perspective view of a single reactant passage of a layer of a microfluidic device, the passage comprising multiple successive chambers of the type shown in FIG. 5A, according to embodiments of the present disclosure; and

FIGS. 6A-6F are schematic views depicting embodiments of a flow-splitting cape comprising a terminus positioned along a central axis within a reactant passage of a layer of a microfluidic device according to embodiments of the present disclosure.

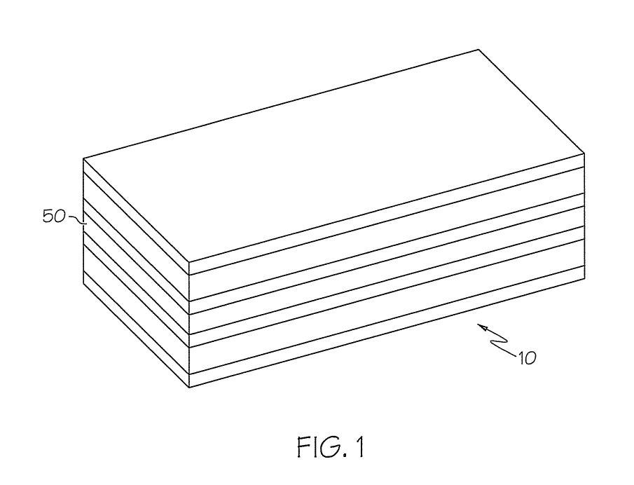

The embodiments set forth in the drawings are illustrative in nature and not intended to be limiting of the invention defined by the claims. Moreover, individual features of the drawings and invention will be more fully apparent and understood in view of the detailed description.

### DETAILED DESCRIPTION

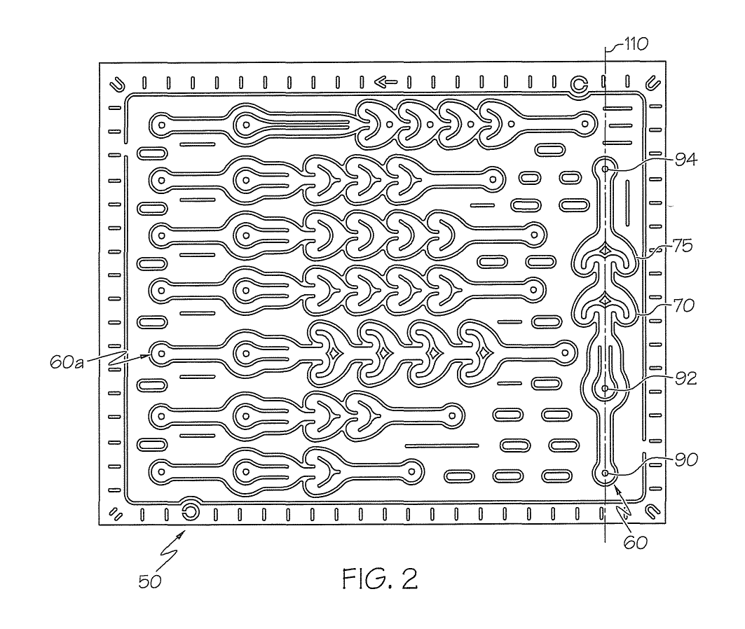

Referring to the embodiment of FIG. 2, a layer 50 of a microfluidic device may comprise at least one reactant passage 60 defined within the layer 50. The reactant passage 60 may be defined by vertical wall structures, of which a cross-section is shown in the figure. As shown, multiple different reactant passages with various profiles may be used within the layer 50. Moreover, while various materials are considered suitable, the layer 50 desirably may be formed of glass, glass-ceramic, ceramic, or mixtures or combinations thereof. Other materials, such as metal or polymer, may also be used if desired.

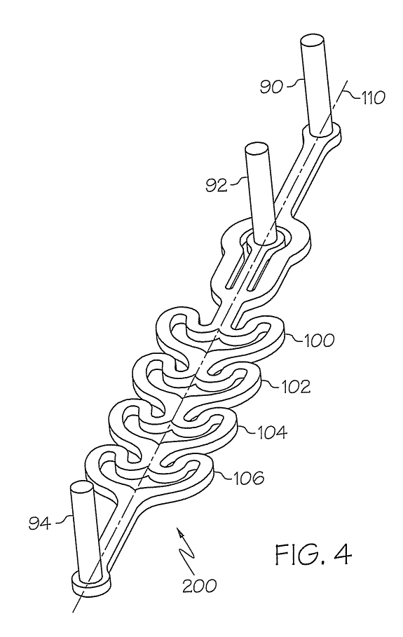

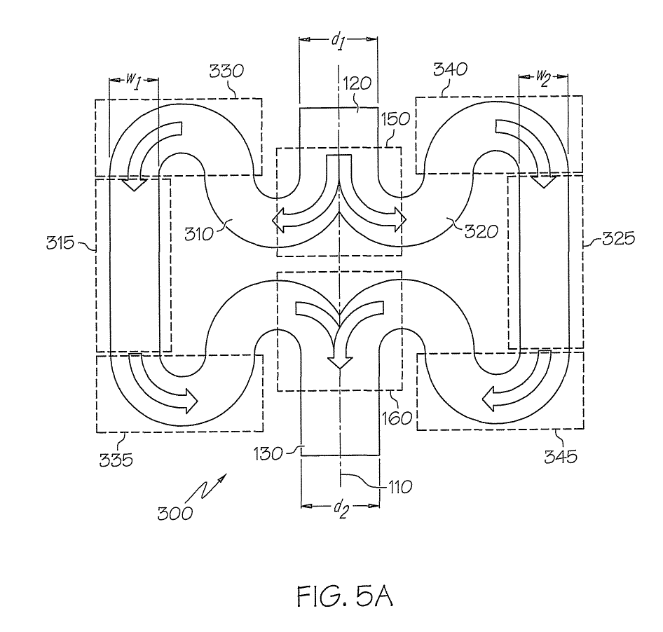

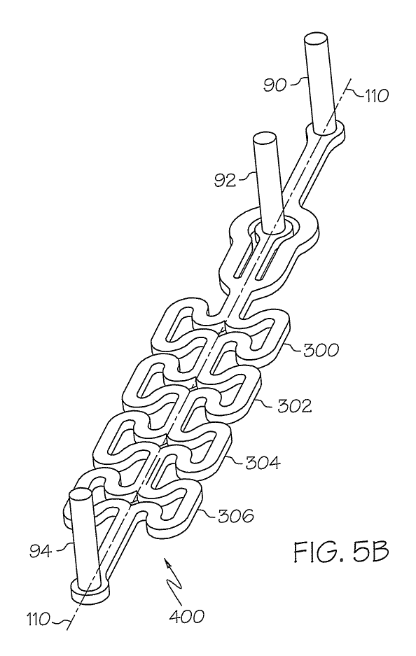

Referring again to FIG. 2, each reactant passage 60 may comprise one or more chambers 70, 75 disposed along a central axis 110. In some embodiments, as shown in the figure a reactant passage 60 may comprise multiple chambers 70, 75 arranged in succession. As used herein, “in succession” with respect to arrangement of multiple chambers means that a chamber outlet (described below) of a first chamber 70 is in fluid communication with a chamber inlet (described below) of a second chamber 75. Though FIG. 2 depicts two chambers 70, 75 in succession, it is contemplated to use only one chamber (not shown) or more than two chambers, such as in passage 60a. As further examples, FIG. 4 depicts a reactant passage 200 comprising four chambers 100, 102, 104, and 106 disposed along central axis 110, and FIG. 5B also displays a four-chamber (300, 302, 304, and 306) reactant passage 400 disposed along central axis 110. Though four chambers are depicted in these figures, it will be understood that a reactant passage according to embodiments of the present disclosure need not be limited to four chambers.

Referring again to FIG. 2, in some embodiments a reactant passage 60 may comprise at least one feed inlet 90, 92, through which fluids are introduced into the reactant passage 60 to be mixed as they flow through chambers 70 and 75. Moreover, the reactant passage 60 may comprise at least one product outlet 94, through which mixed fluids may leave the reactant passage 60. As shown in FIG. 2, the reactant passage 60 may include two inlets 90 and 92 and one outlet 94 disposed near opposite ends of the reactant passage 60; however, it is contemplated to include more or fewer inlets or outlets as well as to arrange the inlets and outlets at different locations on the reactant passage 60.

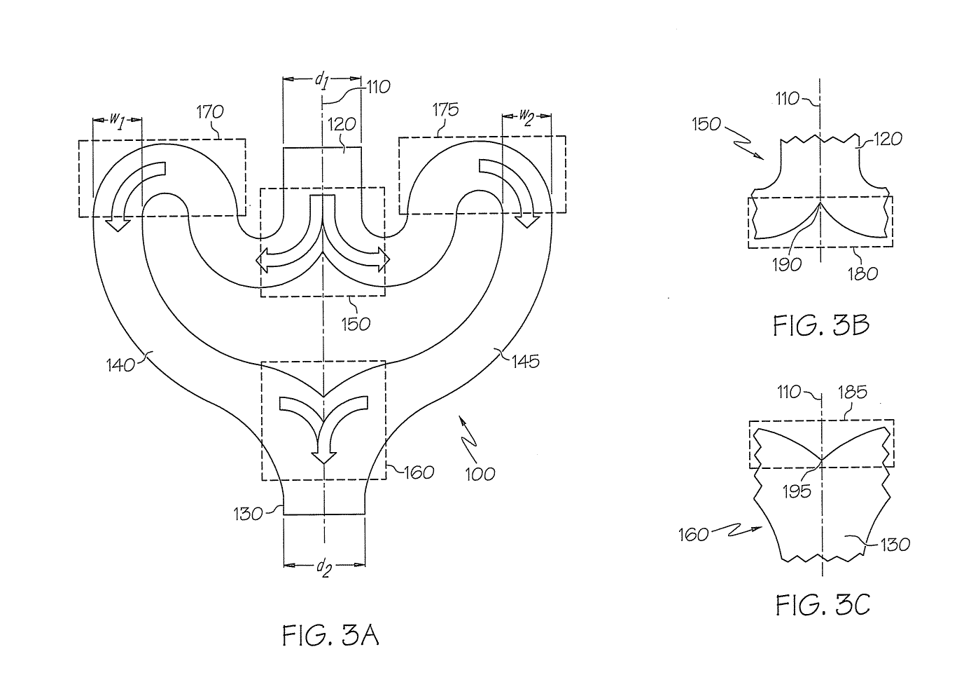

Referring to FIG. 3A, each chamber 100 in the reactant passage may comprise a chamber inlet 120 disposed along the central axis 110, a chamber outlet 130 disposed along the central axis 110, and two subpassages 140, 145 disposed between the chamber inlet 120 and the chamber outlet 130. Each subpassage 140, 145 may define a path that diverges from the central axis 110 and then converges toward the central axis 110. In one embodiment, chamber outlet 130 may comprise a width d₂ that is substantially equal to the width d₁ of chamber inlet 120. In other embodiments, subpassages 140 and 145 may define symmetric paths relative to central axis 110. In some embodiments, subpassages 140 and 145 may be at least partially curved. In some embodiments, subpassages 140 and 145 may comprise a widths w₁ and w₂, both of which are less than the width d₁ of the chamber inlet 120 and the width d₂ of the chamber outlet 130.

Referring yet again to FIG. 3A, subpassages 140 and 145 each may comprise at least one bend, examples of which are shown as 170 and 175. Each bend, for example 170 and 175, may define a shape configured to change the direction of a fluid flowing through the subpassage in which the bend is disposed by at least 90°. As shown in the figure for illustration and not by way of limitation, bends 170 and 175 may be disposed along the path of their respective subpassages 140 and 145 at a position at which the subpassage diverges most greatly from central axis 110. In some embodiments, bends 170 and 175 may be in fluid communication with a curved region of the subpassage 140 and 145 respectively.

Referring to an alternative embodiment as shown in FIG. 5A, the subpassages 310 and 320 each may comprise at least two spaced bends. For example, subpassage 310 comprises two spaced bends, 330 and 335, and subpassage 320 comprises two spaced bends, 340 and 345. In some embodiments, each subpassage may comprise a straight region disposed between any two spaced bends. For example, subpassage 310 comprises a straight region 315 disposed between spaced bends 330 and 335. Similarly, subpassage 320 comprises a straight region 325 disposed between spaced bends 340 and 345. In some embodiments, width w₁ of straight region 315 of subpassage 310 may be substantially equal to width w₂ of straight region 325 of subpassage 320.

Referring yet again to FIGS. 3A-3C, each chamber 100 may comprise further a flow-splitting region 150 disposed between the two subpassages 140, 145 and the chamber inlet 120, such that the flow-splitting region 150 divides the chamber inlet 120 into the two subpassages 140, 145. Furthermore, a flow-joining region 160 may be disposed between the two subpassages 140, 145 and the chamber outlet 130, such that the flow-joining region 160 merges the two subpassages 140, 145. Chamber outlet 130 may be in fluid communication with a chamber inlet of a successive chamber (not shown) within a reactant passage.

Further as shown, each chamber 100 may comprise at least one flow-directing cape in the flow-splitting region 150, the flow-joining region 160, or both. The flow-splitting region 150 may comprise at least one flow-directing cape 180 disposed opposite the chamber inlet 120 and comprising a terminus 190 positioned along the central axis 110. Moreover, the flow-joining region 160 may comprise at least one flow-directing cape 185 disposed opposite the chamber outlet 130 and comprising a terminus 195 positioned along the central axis 110. As shown in FIG. 3B, flow-splitting region 150 may comprise at least one flow-directing cape 180, disposed opposite chamber inlet 120. Flow-directing cape 180 may comprise a terminus 190 positioned along central axis 110. As shown in FIG. 3C, flow-joining region 160 may comprise at least one flow-directing cape 185, disposed opposite chamber outlet 130. Flow-directing cape 185 may comprise a terminus 195 positioned along central axis 110.

As illustrated in FIGS. 3B and 3C, a “flow-directing cape” denotes any flow-directing structure that, when positioned opposite a chamber inlet 120 or a chamber outlet 130, defines a flow-directing cross section that contracts to a flow-directing terminus 190 or 195 as it extends along the central axis 110 in the direction of the chamber inlet 120 or chamber outlet 130, respectively. Though FIG. 3A describes both the flow-splitting region 150 and the flow-joining region 160 as including flow-directing capes 180 and 185, respectively, it is contemplated as stated above that some chambers 100 alternatively may only utilize one flow-directing cape.

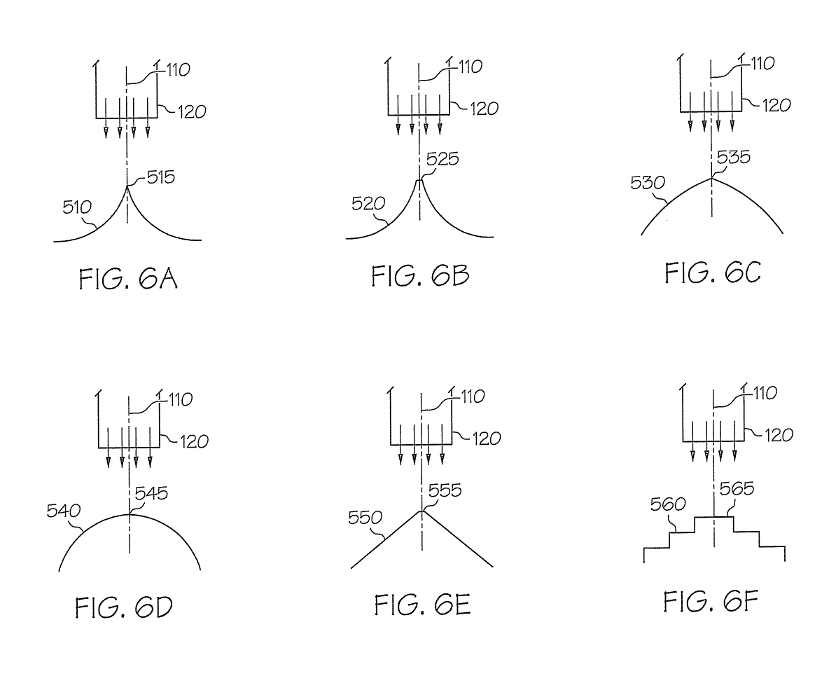

FIGS. 6A-6F each depict, without limitation, various exemplary embodiments of the flow-directing cape structures identified in previous embodiments of the reactant passage chambers. In each of the figures, a chamber inlet 120 is depicted, disposed along a central axis 110. Each flow-directing cape structure is disposed opposite the chamber inlet 120. Each flow-directing cape structure comprises a terminus positioned along central axis 110. Though the downwardly pointing mows represent a fluid flow directed into a chamber and toward the depicted cape structure, it will be understood that identical structures for flow-directing cape structures according to the exemplary embodiments shown in these figures are intended for when the flow direction is reversed (i.e., when fluid flow approaches the flow-directing cape structure from the left and right sides according to the figure and is directed upwardly through a chamber outlet). Furthermore, it will be apparent to someone of ordinary skill in the art that numerous variations and combinations of the embodiments of flow-directing cape structures are possible without departing from the scope of the present invention.

In an exemplary embodiment shown in FIG. 6A, flow-directing cape structure 510 defines an inwardly-curving (concave) profile on both sides of central axis 110 and a single-point terminus 515 at the intersection of the sides of flow-directing cape structure 510 with central axis 110. In another exemplary embodiment shown in FIG. 6B, flow-directing cape structure 520 also defines a concave profile on both sides of central axis 110. As distinguished from the single-point terminus 515, terminus 525 is disposed on a horizontal surface formed from truncating the concave profile of flow-directing cape structure 520. In an alternative embodiment not shown, a flow-directing cape structure could be shaped similar to flow-directing cape structure 520 but with the truncated concave profile replaced by a rounded top portion comprising a terminus.

Referring to FIG. 6C, the flow-directing cape structure 530 defines an outwardly-curving (convex) profile adjacent terminus 535 on both sides of central axis 110. In yet another exemplary embodiment shown in FIG. 6D, the flow-directing cape structure 540 defines a smooth arc profile with terminus 545 disposed on central axis 110. In an alternative embodiment not shown, a flow-directing cape structure could have a terminus disposed on a horizontal surface formed by truncating the convex profile of flow-directing cape structure 535.

In an exemplary embodiment shown in FIG. 6E, flow-directing cape structure 550 is neither concave nor convex but merely slanted. Terminus 555 defines the only point on flow-directing cape structure 550 closest to flow inlet 120. In alternative embodiments, the structure depicted as 550 could be truncated. In an exemplary embodiment shown in FIG. 6F, flow-directing cape structure 560 defines a stepped structure, wherein the terminus 565 constitutes an upper flat surface on the stepped structure.

The microfluidic devices as described through the various embodiments of the present invention are capable of effectively mixing immiscible liquids, emulsions, and gas-liquid dispersions within a microreactor. The microfluidic devices according to embodiments of the present invention may achieve higher throughput by maintaining or raising the quality of fluid mixing and reducing pressure-resistance to fluid flow. Not to be bound by theory, it is believed that the microfluidic devices of the present disclosure provide both increased mixing quality and decreased pressure drop by eliminating deleterious effects such as vortices, general recirculation, and “dead zones” within a microreactor.

The methods and/or devices disclosed herein are generally useful in performing any process that involves mixing, separation, extraction, crystallization, precipitation, or otherwise processing fluids or mixtures of fluids, including multiphase mixtures of fluids—and including fluids or mixtures of fluids including multiphase mixtures of fluids that also contain solids—within a microstructure. The processing may include a physical process, a chemical reaction defined as a process that results in the interconversion of organic, inorganic, or both organic and inorganic species, a biochemical process, or any other form of processing. The following non-limiting list of reactions may be performed with the disclosed methods and/or devices: oxidation; reduction; substitution; elimination; addition; ligand exchange; metal exchange; and ion exchange. More specifically, reactions of any of the following non-limiting list may be performed with the disclosed methods and/or devices: polymerisation; alkylation; dealkylation; nitration; peroxidation; sulfoxidation; epoxidation; ammoxidation; hydrogenation; dehydrogenation; organometallic reactions; precious metal chemistry/homogeneous catalyst reactions; carbonylation; thiocarbonylation; alkoxylation; halogenation; dehydrohalogenation; dehalogenation; hydroformylation; carboxylation; decarboxylation; amination; acylation; peptide coupling; aldol condensation; cyclocondensation; dehydrocyclization; esterification; amidation; heterocyclic synthesis; dehydration; alcoholysis; hydrolysis; ammonolysis; etherification; enzymatic synthesis; ketalization; saponification; isomerisation; quaternization; formylation; phase transfer reactions; silylations; nitrile synthesis; phosphorylation; ozonolysis; azide chemistry; metathesis; hydrosilylation; coupling reactions; and enzymatic reactions.

For the purposes of describing and defining the present invention it is noted that the terms “substantially” and “about” are utilized herein to represent the inherent degree of uncertainty that may be attributed to any quantitative comparison, value, measurement, or other representation. Also, the terms “substantially” and “about” are utilized herein to represent the degree by which a quantitative representation may vary from a stated reference without resulting in a change in the basic function of the subject matter at issue. Moreover, although the term “at least” is utilized to define several components of the present invention, components which do not utilize this term are not limited to a single element. It is noted also that recitations herein of “at least one” component, element, etc., should not be used to create an inference that the alternative use of the articles “a” or “an” should be limited to a single component, element, etc.

The terms “horizontal” and “vertical,” as used in this document are relative terms that do not necessarily indicate perpendicularity. The terms also are used for convenience to refer to orientations used in the figures, which orientations are used as a matter of convention only and are not intended as characteristic of the devices shown. The present invention and the embodiments thereof to be described herein may be used in any desired orientation, and horizontal and vertical walls need be only intersecting walls, not necessarily perpendicular walls.

To the extent that any meaning or definition of a term in this written document conflicts with any meaning or definition of the term in a document incorporated by reference, the meaning or definition assigned to the term in this written document shall govern.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A microfluidic device 10 comprising at least one reactant passage 60 defined within a layer 50 of the microfluidic device 10, each reactant passage 60 comprising one or more chambers 70, 75 disposed along a central axis 110, wherein each chamber comprises:
a chamber inlet 120 disposed along the central axis 110;
a chamber outlet 130 disposed along the central axis 110;
two subpassages 140, 145, each disposed between the chamber inlet 120 and the chamber outlet 130, wherein each subpassage 140, 145 defines a path that diverges from the central axis 110 and then converges toward the central axis 110;
a flow-splitting region 150 disposed between the two subpassages 140, 145 and the chamber inlet 120, wherein the flow-splitting region 150 divides the chamber inlet 120 into the two subpassages 140, 145;
a flow-joining region 160 disposed between the two subpassages 140, 145 and the chamber outlet 130, wherein the flow-joining region 160 merges the two subpassages 140, 145;
wherein the flow-splitting region 150 comprises at least one flow-directing cape 180 disposed opposite the chamber inlet 120, the flow-joining region 160 comprises at least one flow-directing cape 185 disposed opposite the chamber outlet 130, and each flow-directing cape 180, 185 comprises a terminus 190, 195 positioned along the central axis 110.

2. The microfluidic device 10 of claim 1, wherein at least one reactant passage 60 comprises multiple chambers 70, 75 arranged in succession.

3. The microfluidic device 10 of claim 2, wherein the chamber outlet 130 of a first chamber 70 is in fluid communication with a chamber inlet 120 of a successive chamber 75.

4. The microfluidic device 10 of claim 1, wherein each terminus 190, 195 is curved, straight, or combinations thereof.

5. The microfluidic device 10 of claim 1, wherein the chamber outlet 130 comprises a width d₂ substantially equal to a width d₁ of the chamber inlet 120.

6. The microfluidic device 10 of claim 1, wherein the two subpassages 140, 145 are symmetric to one another relative to the central axis 110.

7. The microfluidic device 10 of claim 1, wherein the width of each subpassage 140, 145 is less than the widths d₁, d₂ of the chamber inlet 120 and the chamber outlet 130, respectively.

8. The microfluidic device 10 of claim 1, wherein each subpassage 140, 145 is at least partially curved.

9. The microfluidic device 10 of claim 1, wherein each subpassage 140 comprises at least one bend 170.

10. The microfluidic device 10 of claim 9, wherein each bend 170, 175 defines a shape configured to change the direction of fluid flow by at least 90°.

11. The microfluidic device 10 of claim 9, wherein the bend 170 is disposed along the path of the subpassage 140 at a position where the subpassage 140 diverges most greatly from the central axis 110.

12. The microfluidic device 10 of claim 1, wherein the microfluidic device 10 is formed of one or more of glass, glass-ceramic, and ceramic.

13. The microfluidic device 10 of claim 1, wherein each subpassage 310 comprises at least two spaced bends 330, 335.

14. The microfluidic device 10 of claim 13, wherein each subpassage 310 comprises a straight region 315 disposed between at least two spaced bends 330, 335.

15. The microfluidic device 10 of claim 14, wherein the straight regions 315, 325 of the two subpassages 140, 145 each comprise a substantially equal width w₁, w₂.
